SEQUENCE LISTING (2 sequences)

SEQ ID NO: 1 - protein, 13 aa, Artificial
  Synthetic Peptide
KSRIVPAIPVSLL

SEQ ID NO: 2 - protein, 14 aa, Artificial
  Synthetic Peptide
LRSPIAPVIPVSKK

(19) United States
(12) Patent Application Publication
(10) Pub. No.: US 2006/0014136 A1
(43) Pub. Date: Jan. 19, 2006

(54) Method of screening for protection from microbial infection

(75) Inventors: Monisha Gough Scott, Vancouver (CA); Danika Louise Goosney, Vancouver (CA); Annett Rozek, Port Moody (CA)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Inimex Pharmaceuticals, Inc., Vancouver (CA)

(21) Appl. No.: 11/182,243

(22) Filed: Jul. 14, 2005

Related U.S. Application Data
(60) Provisional application No. 60/587,822, filed on Jul. 14, 2004.

(51) Int. Cl. C07K 7/08; C12Q 1/00; C12P 21/06

(52) U.S. Cl. 435/4; 435/69.1; 435/320.1; 435/325; 530/326; 530/327

(57) ABSTRACT

Screening assays that allow for the identification of agents that modulate host cell response to microbial infection. The methods of the invention provide the ability to measure the reduction in toxicity to the host cell, which is directly related to the ability of a compound to alter the cell's responses and thereby protect the cell from the consequences of infection.

### CROSS REFERENCE TO RELATED APPLICATION(S)

This application claims the benefit of priority under 35 U.S.C. § 119(e) of U.S. Ser. No. 60/587,822, filed Jul. 14, 2004, the entire content of which is incorporated herein by reference.

### FIELD OF THE INVENTION

The present invention relates generally to modulating innate immunity in a subject and to drug discovery related to pathologies resulting from microbial infections. More specifically, the invention relates to methods of screening for agents that modulate host cell response to microbial infection.

### BACKGROUND OF THE INVENTION

Infectious diseases are the leading cause of death worldwide. According to a 1999 World Health Organization study, over 13 million people die from infectious diseases each year. Infectious diseases are the third leading cause of death in North America, accounting for 20% of deaths annually and increasing by 50% since 1980. The success of many medical and surgical treatments also hinges on the control of infectious diseases. The discovery and use of antibiotics has been one of the great achievements of modern medicine. Without antibiotics, physicians would be unable to perform complex surgery, chemotherapy or most medical interventions such as catheterization.

Current sales of antibiotics are US$26 billion worldwide. However, the overuse and sometimes unwarranted use of antibiotics have resulted in the evolution of new antibiotic-resistant strains of bacteria. Antibiotic resistance has become part of the medical landscape. Bacteria such as vancomycin-resistant Enterococcus, VRE, and methicillin-resistant Staphylococcus aureus MRSA strains cannot be treated with antibiotics and often, patients suffering from infections with such bacteria die. Antibiotic discovery has proven to be one of the most difficult areas for new drug development and many large pharmaceutical companies have cut back or completely halted their antibiotic development programs. However, with the dramatic rise of antibiotic resistance, including the emergence of untreatable infections, there is a clear unmet medical need for novel types of anti-microbial therapies, and agents that impact on innate immunity would be one such class of agents.

The host innate immune system is a highly effective and evolved general defense system that recognizes and counters microbial infections. Elements of innate immunity are always present at low levels and are activated very rapidly when stimulated. Stimulation can include interaction of bacterial signaling molecules with pattern recognition receptors on the surface of the body's cells or other mechanisms of disease. Every day, humans are exposed to tens of thousands of potential pathogenic microorganisms through the food and water we ingest, the air we breathe and the surfaces, pets and people that we touch. The innate immune system acts to prevent these pathogens from causing disease. The innate immune system includes a range of protective mechanisms including epithelial barrier function and secretion of cytokine and chemokines. There is evidence to indicate that innate responses are key to controlling most infections, as well as contributing to inflammatory responses. Inflammatory responses triggered by infection are central components of disease pathogenesis. The importance of Toll-like receptors (TLRs) in the innate immune response has been well characterized. The mammalian family of TLRs recognizes conserved molecules, many of which are found on the surface of, or are released by, microbial pathogens. There are also a number of less well characterized mechanisms that initiate and or contribute to the host innate defense.

The innate immune system differs from so-called adaptive immunity (which includes antibodies and antigen-specific B- and T-lymphocytes) because it is always present, effective immediately, and relatively non-specific for any given pathogen. The adaptive immune system requires amplification of specific recognition elements and thus takes days to weeks to respond. Even when adaptive immunity is pre-stimulated by vaccination, it may take three days or more to respond to a pathogen whereas innate immunity is immediately or rapidly (hours) available. Innate immunity involves a variety of effector functions including phagocytic cells, complement, etc, but is generally incompletely understood. Generally speaking many innate immune responses are “triggered” by the binding of microbial signaling molecules with pattern recognition receptors (e.g., Toll-like receptors, scavenger receptors, nucleotide-binding oligomerization domain (NOD) proteins) on the surface of, or within, host cells. Many of these effector functions are grouped together in the inflammatory response. However too severe an inflammatory response can result in responses that are harmful to the body, and in an extreme case sepsis and potentially death can occur.

The release of structural components from infectious agents during infection causes an inflammatory response, which when unchecked can lead to the potentially lethal condition, sepsis. Sepsis occurs in approximately 780,000 patients in North America annually. Sepsis may develop as a result of infections acquired in the community such as pneumonia, or it may be a complication of the treatment of trauma, cancer or major surgery. Severe sepsis occurs when the body is overwhelmed by the inflammatory response and body organs begin to fail. Up to 120,000 deaths occur annually in the United Stated due to sepsis. Sepsis may also involve pathogenic microorganisms or toxins in the blood (e.g., septicemia), which is a leading cause of death among humans. Gram-negative bacteria are the organisms most commonly associated with such diseases. However, gram-positive bacteria are an increasing cause of infections. Gram-negative and Gram-positive bacteria and their components can all cause sepsis.

A variety of microorganisms can cause disease including viruses, bacteria, fungi, and parasites. Microbial cells are distinct from the cells of animals and plants, which are unable to live alone in nature but can exist only as parts of multicellular organisms. Microbial cells can be pathogenic or nonpathogenic. This attribute depends in part on the microorganism and in part on the status of the host. For example, in an immunocompromised host, a normally harmless bacterium can become a pathogen. Host defense against microorganisms begins with the innate immune system, including the epithelial barriers of the body, and culminates in the induction of the adaptive immune response. Entry into host cells is critical for the survival of bacterial pathogens that replicate in an intracellular milieu. For organisms that replicate at extracellular sites, the significance of bacterial entry into host cells is less well defined.

Pseudomonas aeruginosa is a Gram-negative bacterium that is noted for its environmental versatility, ability to cause disease in particular susceptible individuals, and its resistance to antibiotics. It is a versatile organism that grows in soil, marshes, and coastal marine habitats, as well as on plant and animal tissues. The most serious complication of cystic fibrosis is respiratory tract infection by the ubiquitous bacterium P. aeruginosa. Cancer and burn patients also commonly suffer serious infections by this organism, as do certain other individuals with immune systems deficiencies. Unlike many environmental bacteria, P. aeruginosa has a remarkable capacity to cause disease in susceptible hosts.

Staphylococcus aureus is a Gram positive spherical bacteria, about 1 micrometer in diameter, that occurs in microscopic clusters and causes a range of infections from endocarditis to pneumonia. It is one of the most important human pathogens, which causing both community-acquired and nosocomial infections. Although S. aureus is generally classified as an extracellular pathogen, recent data has revealed its ability to infect various types of host cells: both professional phagocytes and nonphagocytes, including endothelial cells, fibroblasts, and others. This invasion is initiated by the adherence of S. aureus to the cell surface, a process in which staphylococcal fibronectin-binding proteins play a prominent role. Phagocytosed S. aureus can either induce apoptosis of the host cell or survive for several days in the cytoplasm, which is thought to be devoid of anti-staphylococcal effector mechanisms. It is still unclear whether invasion and cytotoxicity are a common feature of clinical S. aureus isolates and whether these factors contribute to pathogenicity. S. aureus colonize nasal passages, skin surfaces, mucous membranes and areas around the mouth, genitals, and rectum. S. aureus may cause include superficial skin lesions such as boils, styes, and furuncles. More serious infections include pneumonia, mastitis, phlebitis, meningitis, and urinary tract infections, whereas deep-seated infections include osteomyelitis and endocarditis. Penicillin was effective in treating S. aureus until the bacterium became resistant. Throughout the second half of the 20th century, new antibiotics such as vancomycin and methicillin were developed which successfully cured S. aureus infections. However, methicillin-resistant strains of S. aureus evolved in the 1970s and have troubled hospitals worldwide with persistent infections in their patients ever since. More recently vancomycin-resistant strains of S. aureus have surfaced.

Klebsiella pneumoniae is a large, non-motile gram-negative bacterium. It is an opportunistic pathogen that frequently causes nosocomial infections, mainly in immunocompromised patients. K. pneumoniae infections range from mild urinary infections to severe bacteremia and pneumonia with a high rate of mortality and morbidity. Pulmonary infections due to K. pneumoniae are often characterized by a rapid progressive clinical course complicated by lung abscesses and mulitiulobular involvement which leaves little time to establish an effective antibiotic treatment. Klebsiella infections are encountered far more often now than in the past. This is probably due to the bacterium's antibiotic resistance properties. They may contain resistance plasmids (R-plasmids) which transfer resistance to such antibiotics as ampicillin and carbenicillin to others of the same species. Moreover, the R-plasmids can be transferred to other enteric bacteria not necessarily of the same species. The mortality for gram-negative pneumonia is about 25 to 50% despite the availability of effective antibiotics. Examples of the effective antibiotics that are used to treat K. pneumoniae patients are cephalosporin, imipenem, or ciprofloxacin. Each of these drugs can be given alone or in combination with an aminoglycoside but aminoglycosides should not be used alone. A broad-spectrum cephalosporin may be used alone although this may cause the risk of emerging resistance during treatment, primarily with P. aeruginosa.

Salmonella species including Salmonella Typhimurium are Gram-negative, flagellated, facultatively anaerobic bacilli. Salmonellosis ranges clinically from the common Salmonella gastroenteritis (diarrhea, abdominal cramps, and fever) to enteric fevers (including typhoid fever). Non-typhoidal Salmonella strains include S. enteriditis, S. typhimurium, S. newport and S. anatum, and account for 10-15% of all cases of food poisoning in North America. Non-typhoidal salmonellosis is a worldwide disease of humans and animals. Animals are the main reservoir, and the disease is usually food borne such as from chicken meat or eggs, although it can be spread from person to person. The incubation period for Salmonella depends on the dose of bacteria. Symptoms usually begin 6 to 48 hours after ingestion of contaminated food or water. There are effective vaccines for typhoid fever but not for non-typhoidal salmonellosis. Salmonella induces host cell membrane ruffling in order to enter host cells. Ruffling occurs non-specifically and wraps around the bacteria, pulling them into the cells. In the cells, Salmonella end up in membrane-bound vesicles called Salmonella-containing vacuoles (SCV).

In the last decade, microorganisms previously believed to be relatively harmless have emerged as among the most dangerous. The fungal pathogen, Candida albicans, is a daunting example of this problem. For many individuals, this fungus can be routinely found on skin, in genitourinary sites, and in the mouth. For those suffering from conditions or treatments that compromise immunity, including chemotherapy, renal dialysis, organ transplantation, diabetes, or HIV infection, Candida proliferates and gains access to deeper tissues, where it may reach the bloodstream only to be disseminated to cause infection throughout the body. Adherence to cells lining human blood vessels triggers a metamorphosis in this pathogen, providing it new weapons to penetrate into deeper tissues and organs. Unless Candida can adhere to such cells, it is quickly cleared from the bloodstream, and rendered harmless.

It is desired to find new compounds that act on the host and not on the pathogen. Normal mechanisms of microbial resistance against antibiotics are unlikely to be observed in response to treatment with these compounds. For example, rapid evolution of bacterial resistance through plasmid exchange will not be promoted through the use of compounds that do not act on the bacteria

### SUMMARY OF THE INVENTION

The present invention is based on the seminal discovery that based on a host cell's response to microbial infection, one can screen for novel compounds that modulate or decrease the toxicity of the microbial infection to the cell.

Thus, in one embodiment, a method of identifying agents that modulate host cell response to microbial pathogens is provided. The method includes contacting a host cell with a microbial pathogen under conditions sufficient to cause a response. Such a response includes, but is not limited to, cell damage or cell death. Thereafter, the host cell is brought into contact with a test agent suspected of modulating host cell response to the microbial pathogen. Detecting a change in host cell response in the presence of the test agent, as compared to the host cell response in the absence of the test agent, identifies the test agent as an agent that modulates host cell response to microbial pathogens. Examples of microbial pathogens include bacteria, fungi, viruses, and parasites. The host cell may be mammalian or any other cell susceptible to microbial infection. The methods of the invention are preferably run with test agents that are non-anti-microbial to eliminate the possibility of direct action on the microbial pathogen.

In another embodiment, the invention provides a method for stimulating innate immunity in a subject. The method includes administering to a subject suffering from microbial infection an agent identified as modulating host cell response to microbial pathogens. In this method, the test agent is identified as stimulating innate immunity in the host.

In another embodiment, the invention provides a method of modulating a host cell's response to microbial pathogens comprising contacting the host cell with an agent identified as modulating host cell response to microbial pathogens.

### BRIEF DESCRIPTION OF THE FIGURES

FIG. 1 is a graphical representation of sample assay data with A549 cells, S. aureus and a test compound.

FIG. 2 is a graphical representation of sample assay data with A549 cells, P. aeruginosa and a test compound.

FIG. 3 is a graphical representation of sample assay data with differentiated THP-1 cells, S. aureus and a test compound.

FIG. 4 is a graphical representation of sample assay data with A549 cells, K. pneumoniae and a test compound

FIG. 5 is a graphical representation of sample assay data with A549 cells, Salmonella Typhimurium and a test compound.

FIG. 6 is a graphical representation of sample assay data with RAW 264.7 cells, S. aureus, and a test compound.

FIG. 7 is a graphical representation of data from in vivo assay demonstrating correlation of in vivo test compound activity with activity in in vitro assay.

### DETAILED DESCRIPTION OF THE INVENTION

The present invention provides novel protection assays to determine the ability of a compound to reduce the cytotoxicity associated with microbial infection.

“Innate immunity” as used herein refers to the natural ability of an organism to defend itself against invasions by pathogens. Pathogens or microbes as used herein, may include, but are not limited to bacteria, fungi, parasite, and viruses. Innate immunity is contrasted with acquired/adaptive immunity in which the organism develops a defensive mechanism based substantially on antibodies and/or immune lymphocytes that is characterized by specificity, amplifiability and self vs. non-self discrimination. With innate immunity, broad, nonspecific immunity is provided and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). In addition, innate immunity includes immune responses that affect other diseases, such as cancer, inflammatory diseases, multiple sclerosis, various viral infections, and the like.

Most bacterial pathogens are present in the general environment or in the host's normal bacterial flora. Bacteria have evolved the ability to cause severe disease by acquiring different mechanisms (called virulence factors) which enable them to colonize, disseminate within and invade host tissues. When these pathogenicity factors are suppressed, bacteria are no longer able to maintain themselves in host tissues, and thus cannot cause disease.

Fungi include moulds, yeasts and higher fungi. All fungi are eukaryotic and have sterols but not peptidoglycan in their cell membrane. Fungal infections or mycoses are classified depending on the degree of tissue involvement and mode of entry into the host. In the immunocompromised host, a variety of normally mild or nonpathogenic fungi can cause potentially fatal infections.

Viruses are unlike fungi and bacteria, lacking many of the attributes of free-living cells. A single virus particle is a static structure, quite stable and unable to change or replace its parts. Only when associated with a cell does a virus become able to replicate and acquire some of the attributes of a living system. Viruses cause numerous diseases including upper respiratory tract infections (URTIs) such as the common cold and pharyngitis (sore throat). Other examples are influenza, gastroenteritis (especially in children), measles, rubella, mumps, chickenpox, glandular fever, cold sores, SARS and AIDS.

Parasites are organisms that derive nourishment and protection from other living organisms known as hosts. They may be transmitted from animals to humans, from humans to humans, or from humans to animals. Several parasites have emerged as significant causes of foodborne and waterborne disease. They may be transmitted from host to host through consumption of contaminated food and water, or by putting anything into your mouth that has touched the stool (feces) of an infected person or animal. These organisms live and reproduce within the tissues and organs of infected human and animal hosts, and are often excreted in feces. Parasites are of different types and range in size from tiny, single-celled, microscopic organisms (protozoa) to larger, multi-cellular worms (helminths) that may be seen without a microscope. Some common parasites are Giardia duodenalis, Cryptosporidium parvum, Cyclospora cayetanensis, Toxoplasma gondii, Trichinella spiralis, Taenia saginata (beef tapeworm), and Taenia solium (pork tapeworm).

To defeat the innate and the adaptive immune system of the host, pathogens such as S. aureus employ both single-gene-encoded virulence factors such as alpha-toxin, coagulase, and protein A, as well as complex mechanisms such as adhesion or slime production. S. aureus expresses many potential virulence factors: (1) surface proteins that promote colonization of host tissues; (2) invasins that promote bacterial spread in tissues (leukocidin, kinases, hyaluronidase); (3) surface factors that inhibit phagocytic engulfment (capsule, Protein A); (4) biochemical properties that enhance their survival in phagocytes (carotenoids, catalase production); (5) immunological disguises (Protein A, coagulase); and (6) membrane-damaging toxins that lyse eukaryotic cell membranes (hemolysins, leukotoxin, leukocidin); (7) exotoxins that damage host tissues or otherwise provoke symptoms of disease (SEA-G, TSST, ET); (8) inherent and acquired resistance to antimicrobial agents.

Microbial pathogens use a number of genetic strategies to invade the host and cause infection. These common themes are found throughout microbial systems. Secretion of enzymes, such as phospholipase, has been proposed as one of these themes that are used by bacteria, parasites, and pathogenic fungi. The role of extracellular phospholipase as a potential virulence factor in pathogenic fungi, including Candida albicans, Cryptococcus neoformans, and Aspergillus, has gained credence recently.

In innate immunity, the immune response is not dependent upon antigens. The innate immunity process may include anatomical barrier function and the production of secretory molecules and cellular components as set forth above. In innate immunity, the pathogens are recognized by receptors encoded in the germline. These receptors (e.g., Toll-like receptors) have broad specificity and are capable of recognizing many pathogens. This change in the immune response induces the release of chemokines, which promote the recruitment of immune cells to the site of infection.

In one aspect, the present invention provides a screening assay to determine the ability of a compound to reduce the toxicity of microbial infection to a host cell. Traditional assays concentrate on direct measurement of anti-microbial action. However, in order to develop drugs capable of enhancing innate immunity by acting only on the host, new assay approaches are required. In this aspect of the invention, bacteria or other microbes are contacted with the cells and incubated for sufficient time to simulate an in vivo microbial infection. This infection causes either cell damage or cell death, which can be measured through a variety of standard assay techniques (eg., LDH assay). Innate immune enhancing drugs are applied to the host cell and any reduction in the toxicity due to the infection is measured. Termed a “protection” assay, the ability to measure the reduction in toxicity is directly related to the ability of a compound to alter the cell's responses and thereby protect the cell from the consequences of infection. The assay is run on compounds demonstrated to be non-anti-microbial, thereby eliminating the possibility of direct action on the microbe.

As used herein, the terms “toxicity” and “cytotoxicity” refer to the quantification of cell death and cell lysis as a result of exposure to a toxic agent or compound. Methods of evaluating toxicity are known in the art.

For example, in one embodiment, toxicity may be evaluated by measuring LDH activity released from the cytosol of damaged cells into the supernatant. LDH is a stable cytosolic enzyme present in all mammalian cells. The normal plasma membrane is impermeable to LDH but damage to the cell membrane results in a change in the membrane permeability and subsequent leakage of LDH into the extracellular fluid. An increase in the amount of dead or plasma membrane-damaged cells results in an increase of the LDH enzyme activity which is measured in the culture supernatant using a simple colorimetric assay.

In another embodiment, toxicity is determined using an MTT assay. The MTT [3-(4,5-dimethylthiazol-2-yl)-2,5-diphenyltetrazolium bromide] assay is based on the ability of a mitochondrial dehydrogenase enzyme from viable cells to cleave the tetrazolium rings of the pale yellow MTT and form dark blue formazan crystals, which are largely impermeable to cell membranes, thus resulting in its accumulation within healthy cells. Solubilization of the cells by the addition of a detergent results in the liberation of the crystals that are solubilized. The number of surviving cells is directly proportional to the level of the formazan product created. The color can then be quantified using a colorimetric assay. The results can be read on a multiwell scanning spectrophotometer (ELISA reader). (Mosmann T. Rapid colorimetric assay for cellular growth and survival: application to proliferation and cytotoxicity assays. J Immunol Methods. 1983 Dec. 16; 65(1-2):55-63).

In another embodiment, toxicity is determined using an adenylate kinase (ToxiLight) bioluminescent assay. This non-destructive cytolysis assay is designed to measure the release of the enzyme, adenylate kinase (AK), from damaged cells. AK is a robust protein present in all eukaryotic cells, which is released into the culture medium when cells die. The enzyme actively phosphorylates ADP to form ATP and the resultant ATP is then measured using the bioluminescent firefly luciferase reaction. As the level of cytolysis increases, the amount of AK in the supernatant also increases, which results in emission of higher light intensity by the ToxiLight reagent.

In another embodiment, toxicity is measured with a Calcein AM assay. The Calcein AM/EthD-III Viability/Cytotoxicity Assay allows simultaneous detection of live cells and dead cells in the same population. Calcein AM specifically stains live cells via their intracellular esterase activity and EthD-III specifically stains dead cells that have lost plasma membrane integrity.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, and the like to produce structural analogs. Candidate agents are also found among biomolecules including, but not limited to: peptides, peptidiomimetics, saccharides, fatty acids, steroids, purines, pyrimidines, polypeptides, polynucleotides, chemical compounds, derivatives, structural analogs or combinations thereof.

Incubating components of a screening assay includes conditions which allow contact between the test compound and the host cell. Contacting includes in solution and in solid phase, or within a cell. The test compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a compound.

“Host cells” or “Recipient cells” encompassed by of the invention are any cells that react to infection by bacteria or other microbes, which may be used in the methods of the invention. The term also includes any progeny of a recipient or host cell, so long as cell death or cell damage may be measured by means of any variety of standard assay techniques known in the art.

Microbial infection in vivo can be a process that occurs at the cellular level and thus can be studied in vitro. The in vitro cytotoxicity of a test compound in cell lines of different sensitivities to toxic effects may be assumed to parallel the range of toxicity occurring as a result of in vivo microbial infection in animal models. The in vitro concentration of the test compound in μg/ml remains constant throughout the experiment and may be empirically expressed as an in vivo dosage in mg/kg/day. FIG. 7 shows the correlation of in vitro assay data to an in vivo infection model. The murine model of bacterial infection was established with a S. aureus strain from the ATCC (Accession No. 25293). In this model, immunocompetent mice were injected with a mucin solution containing the bacterial suspension to facilitate establishment of infection. The severity of disease in this model was assessed by evaluating the bacterial load in peritoneal lavage fluid (usually 24 hours after infection) in addition to clinical observations of the animals. In this intraperitoneal (ip) infection model, test compounds were administered ip at 24 mg/kg.

The invention will now be described in greater detail by reference to the following non-limiting examples. While the invention has been described in detail with reference to certain preferred embodiments thereof, it will be understood that modifications and variations are within the spirit and scope of that which is described and claimed.

### EXAMPLE 1

### Assay Development

The acute in vitro toxicity of the compounds was measured using the Cytotoxicity Detection Kit (Lactate dehydrogenase—LDH) Assay (Roche). This is a calorimetric method for the quantification of cell death and cell lysis, based on the measurement of LDH activity released from the cytosol of damaged cells into the supernatant. LDH is a stable cytosolic enzyme present in all mammalian cells. The normal plasma membrane is impermeable to LDH but damage to the cell membrane results in a change in the membrane permeability and subsequent leakage of LDH into the extracellular fluid. An increase in the amount of dead or plasma membrane-damaged cells results in an increase of the LDH enzyme activity which is measured in the culture supernatant.

Test compounds include, but are not limited to:

| | |
|---|---|
| KSRIVPAIPVSLL - NH₂ | (SEQ ID NO: 1) |
| LRSPIAPVIPVSKK - NH₂ | (SEQ ID NO: 2) |

where NH₂ denotes C-terminal amidation.
A549 Cells and S. aureus.

A549 cells (ATCC CCL 185) were seeded at a density of 1×10⁵ cells/ml 16-20 hours before treatment with peptide. An overnight culture of S. aureus was grown from a single colony on an LB agar plate. The following day, the bacterial culture was subcultured and grown to an OD600 of 0.4-0.6 prior to addition to the cells. The culture medium was removed from the A549 cells and replaced with fresh DMEM (Gibco) containing 10% fetal bovine serum (FBS; Sigma) and the test compounds.

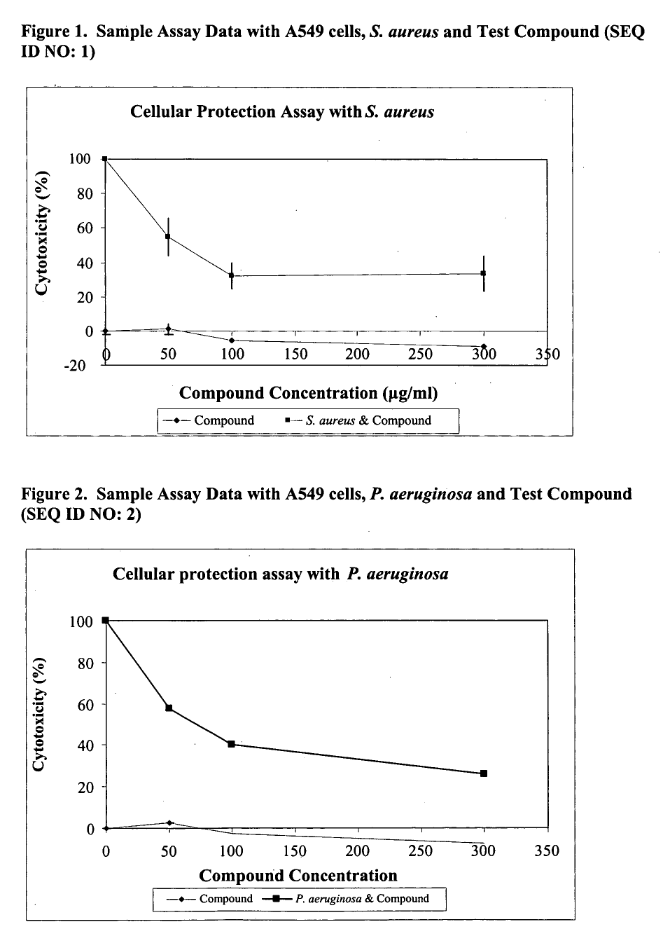

The compounds were added to the A549 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the A549 cells, the subcultured S. aureus was added (m.o.i. approximately 5) for an 18 hour infection. The next day the culture medium was removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 1). The protection from S. aureus induced toxicity was calculated from the absorbance readings at 492 nm by the formula:

$$\frac{( Peptide + S . aureus ( test ) - No Peptide ( neg . control ) )}{( S . aureus ( pos . control ) - No Peptide ( neg . control ) )} \times 100 = \% Toxicity$$

A549 Cells and P. aeruginosa

A549 cells (ATCC CCL 185) were seeded at a density of 1×10⁵ cells/ml 16-20 hours before treatment with peptide. An overnight culture of P. aeruginosa was grown from a single colony on an LB agar plate. The following day, the bacterial culture was subcultured and grown to an OD600 of 0.2-0.4 prior to addition to the cells. The culture medium was removed from the A549 cells and replaced with fresh DMEM (Gibco) containing 10% fetal bovine serum (FBS; Sigma) and the test compounds.

The compounds were added to the A549 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the A549 cells, the subcultured P. aeruginosa was added (m.o.i.˜5) for an 18 hour infection. The next day the culture medium was removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 2). The protection from P. aeruginosa induced toxicity was calculated from the absorbance readings at 492 nm by the formula provided above.

A549 Cells and K. pneumoniae

A549 cells (ATCC CCL 185) were seeded at a density of 1×10⁵ cells/ml 16-20 hours before treatment with peptide. An overnight culture of K. pneumoniae was grown from a single colony on an LB agar plate. The following day, the bacterial culture was subcultured and grown to an OD600 of 0.3-0.5 prior to addition to the cells. The culture medium was removed from the A549 cells and replaced with fresh DMEM (Gibco) containing 10% fetal bovine serum (FBS; Sigma) and the test compounds.

The compounds were added to the A549 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the A549 cells, the subcultured K. penumoniae was added (m.o.i.˜5) for an 5-6 hour infection. The culture medium was then removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 4). The protection from K. pneumoniae induced toxicity was calculated from the absorbance readings at 492 nm by the formula provided above.

A549 Cells and Salmonella Typhimurium

A549 cells (ATCC CCL 185) were seeded at a density of 1×10⁵ cells/ml 16-20 hours before treatment with peptide. An overnight culture of Salmonella Typhimurium was grown from a single colony on an LB agar plate. The following day, the bacterial culture was subcultured and grown to an OD600 of 0.2 prior to addition to the cells. The culture medium was removed from the A549 cells and replaced with fresh DMEM (Gibco) containing 10% fetal bovine serum (FBS; Sigma) and the test compounds.

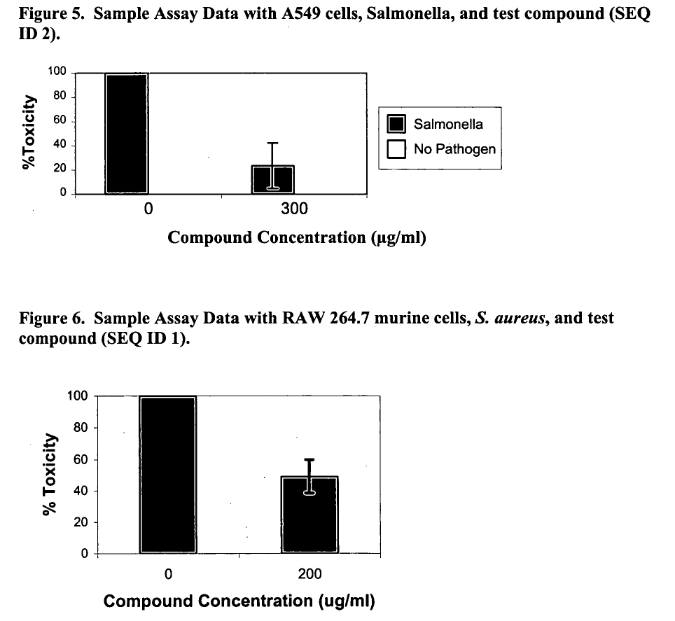

The compounds were added to the A549 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the A549 cells, the subcultured Salmonella Typhimurium was added (m.o.i.=2) for a 6 hour infection. The culture medium was then removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 5). The protection from Salmonella Typhimurium induced toxicity was calculated from the absorbance readings at 492 nm by the formula provided above

A549 Cells and E. coli

A549 cells (ATCC CCL 185) were seeded at a density of 1×10⁵ cells/ml 16-20 hours before treatment with peptide. An overnight culture of E. coli was grown from a single colony on an LB agar plate. The following day, the bacterial culture was subcultured and grown to a range of OD600 values prior to addition to the cells. The culture medium was removed from the A549 cells and replaced with fresh DMEM (Gibco) containing 10% fetal bovine serum (FBS; Sigma) and the test compounds.

The compounds were added to the A549 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the A549 cells, the subcultured E. coli was added (m.o.i.=1-10) for an 18 hour infection. The next day the culture medium was removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells. The protection from E. coli induced toxicity was calculated from the absorbance readings at 492 nm by the formula provided above.

THP-1 Cells and S. aureus

THP-1 human monocytic cells (differentiated* or non-differentiated) were seeded at a density of 1×10⁵ cells/ml 16-20 hours before treatment with peptide. An overnight culture of S. aureus was grown from a single colony on an LB agar plate. The following day, the bacterial culture was subcultured and grown to an OD600 of 0.4-0.6 prior to addition to the cells. The culture medium was removed from the THP-1 cells and replaced with fresh DMEM (Gibco) containing 10% fetal bovine serum (FBS; Sigma) and the test compounds.
*For differentiated THP-1 cells, cells were seeded at a density of 1×10⁵ cells/ml together with phorbol myristate-acetate (100 nM, Sigma) for 24 hours. Following this incubation, cells were washed with DMEM and left to rest for 48 hours in fresh DMEM and 10% FBS prior to addition of test compound.

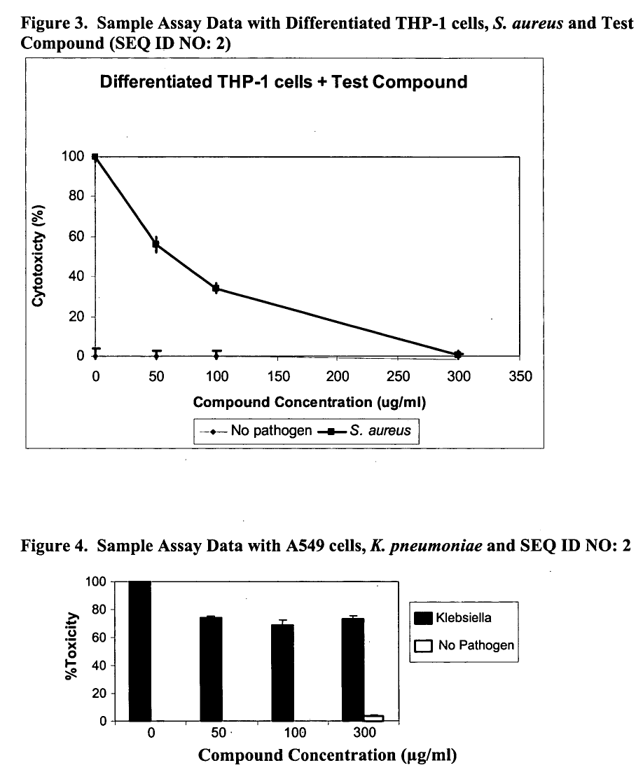

The compounds were added to the THP-1 cells at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the THP-1 cells, the subcultured S. aureus was added (m.o.i.˜5) for an 18 hour infection. The next day the culture medium was removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 3). The protection from S. aureus induced toxicity was calculated from the absorbance readings at 492 nm by the formula provided above.

RAW 264.7 Cells and S. aureus

RAW 264.7 murine macrophage cell line were seeded at a density of 1×10⁵ cells/ml 16-20 hours before treatment with peptide. An overnight culture of S. aureus was grown from a single colony on an LB agar plate. The following day, the bacterial culture was subcultured and grown to an OD600 of 0.3-0.5 prior to addition to the cells. The culture medium was removed from the RAW 264.7 cells and replaced with fresh DMEM (Gibco) containing 10% fetal bovine serum (FBS; Sigma) and the test compounds.

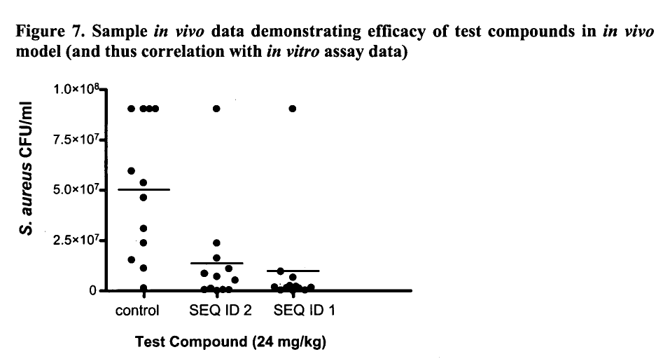

The compounds were added to the RAW 264.7 culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the RAW 264.7 cells, the subcultured S. aureus was added (m.o.i.˜5) for a 6 hour infection. The culture medium was then removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells (see FIG. 6). The protection from S. aureus induced toxicity was calculated from the absorbance readings at 492 nm by the formula provided above.

HBE Cells and S. aureus

HBE (human bronchial epithelial) cells were seeded at a density of 1×10⁵ cells/ml 16-20 hours before treatment with peptide. An overnight culture of S. aureus was grown from a single colony on an LB agar plate. The following day, the bacterial culture was subcultured and grown to an OD600 of 0.5 prior to addition to the cells. The culture medium was removed from the HBE cells and replaced with fresh DMEM (Gibco) containing 10% fetal bovine serum (FBS; Sigma) and the test compounds.

The compounds were added to the HBE culture at a range of concentrations (e.g., 100 μg/ml) and allowed to incubate for 30 minutes at 37° C. and 5% CO₂. Untreated cells were used as a negative control and cells treated with Triton X-100 (Sigma) were used as a positive control for cell toxicity. Following the addition of drug to the HBE cells, the subcultured S. aureus was added (m.o.i.˜5) for an 18 hour infection. The next day the culture medium was removed and tested for the presence of LDH. To test for LDH, 50 μl of cell supernatant was incubated with LDH substrate in a 96-well plate in the dark for 30 minutes at room temperature. The absorbance was measured at 492 nm with a spectrophotometer. The intensity of colour formed in the assay is proportional to the number of lysed cells. The protection from S. aureus induced toxicity was calculated from the absorbance readings at 492 nm by the formula provided above.

A549 Cells and S. pneumoniae

A549 cells (ATCC CCL 185) will be seeded at a density of 1×10⁵ cells/ml 16-20 hours before treatment with peptide. An overnight culture of S. pneumoniae will be grown from a single colony on an LB agar plate. The bacterial culture will be subcultured and grown to a range of OD600 values prior to addition to the cells. The culture medium will be removed from the A549 cells and replaced with fresh DMEM (Gibco) containing 10% fetal bovine serum (FBS; Sigma) and the test compounds.

The compounds will be added to the A549 culture at a range of concentrations (e.g., 100 μg/ml) and allowed to incubate for about 30 minutes at 37° C. and 5% CO₂. Untreated cells will be used as a negative control and cells treated with Triton X-100 (Sigma) will be used as a positive control for cell toxicity. Following the addition of drug to the A549 cells, the subcultured S. pneumoniae will be added (m.o.i.˜1-10) for a 6-18 hour infection. The culture medium will then be removed and tested for the presence of LDH. To test for LDH, 50 μl of cell supernatant will be incubated with LDH substrate in the dark for about 30 minutes at room temperature. The absorbance will be measured at 492 nm with a spectrophotometer. The intensity of colour formed in the assay will be proportional to the number of lysed cells. The protection from S. pneumoniae induced toxicity will be calculated from the absorbance readings at 492 nm by the formula provided above.

Although the invention has been described with reference to the presently preferred embodiment, it should be understood that various modifications can be made without departing from the spirit of the invention. Accordingly, the invention is limited only by the following claims.

What is claimed is:

1. A method of screening for agents that modulate host cell response to microbial pathogens comprising:
(a) contacting a host cell with a microbial pathogen under conditions sufficient to cause a response;
(b) contacting the host cell with a test agent suspected of modulating host cell response to the microbial pathogen;
(c) detecting a change in host cell toxicity in the presence of the test agent as compared to host cell toxicity in the absence of the test agent,
wherein a change in host cell toxicity identifies the test agent as an agent that modulates host cell response to microbial pathogens.

2. The method of claim 1, wherein the microbial pathogen is a bacterium, fungus, virus, or parasite.

3. The method of claim 1, wherein the host cell is a mammalian cell.

4. The method of claim 1, wherein the test agent is non-anti-microbial.

5. The method of claim 1, wherein the test agent stimulates innate immunity in a host.

6. A method for stimulating innate immunity in a subject comprising administering to a subject an agent identified by the method of claim 1.

7. A method of modulating a host cell's response to microbial pathogens comprising contacting the host cell with an agent identified by the method of claim 1.

8. An isolated peptide, wherein the peptide is SEQ ID NO: 1 or 2.
